SEQUENCE LISTING (6 sequences)

SEQ ID NO: 1 - protein, 647 aa, Homo sapiens
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSP

SEQ ID NO: 2 - protein, 355 aa, Homo sapiens
SGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPG

SEQ ID NO: 3 - protein, 681 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSP

SEQ ID NO: 4 - protein, 387 aa, Homo sapiens
MTPGTQSPFFLLLLLTVLTATTAPKPATVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPG

SEQ ID NO: 5 - protein, 257 aa, Borrelia burgdorferi
CKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 6 - protein, 273 aa, Borrelia burgdorferi
MKKYLLGIGLILALIACKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

(19) United States
(12) Patent Application Publication
(10) Pub. No.: US 2021/0046130 A1
(43) Pub. Date: Feb. 18, 2021

(54) RECOMBINANT VIRAL VACCINES

(71) Applicant: VIROGIN BIOTECH CANADA LTD, Vancouver (CA)

(72) Inventors: William Jia, Burnaby (CA); Dmitry V. Chouljenko, Vancouver (CA); Yanal M. Murad, Vancouver (CA); Xiaohu Liu, Vancouver (CA)

(21) Appl. No.: 16/964,429

(22) PCT Filed: Jan. 24, 2019
(86) PCT No.: PCT/US19/15033

§ 371 Date: Jul. 23, 2020

Related U.S. Application Data
(60) Provisional application No. 62/621,468, filed on Jan. 24, 2018.

(51) Int. Cl. A61K 35/76; A61K 38/19; A61K 38/20; C12N 15/86

(52) U.S. Cl. CPC A61K 35/76; A61K 38/191; A61K 2039/6075; A61K 38/2086; C12N 15/86; A61K 38/208

(57) ABSTRACT

Vaccines are provided comprising a recombinant virus which expresses an immunomodulatory protein and a target antigen unrelated to said recombinant virus, and a pharmaceutically acceptable excipient.

### CROSS-REFERENCE TO RELATED APPLICATION

This patent application claims the benefit under 35 U.S.C. § 119(e) of U.S. Provisional Patent Application No. 62/621,468 filed Jan. 24, 2018, which application is incorporated herein by reference in its entirety for all purposes.

### FIELD OF THE INVENTION

The present invention relates generally to vaccines, and more specifically, to recombinant viral vectors which express an immunomodulatory protein and a target antigen unrelated to said recombinant viral vector.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “VIR0408_ST25.txt”, a creation date of Jan. 22, 2019, and a size of 22.2 KB. The Sequence Listing filed via EFS-Web is part of the specification and is incorporated in its entirety by reference herein.

### BACKGROUND

Vaccines or vaccination, the administration of an antigen to stimulate an immune response to a pathogenic agent, have been available for hundreds of years. Smallpox, which is believed to have appeared around 10,000 BC, was a scourge of many ancient societies. Over centuries it had become known that survivors of smallpox became immune to the disease and were called upon to nurse those sick with the disease. One successful way for preventing smallpox that eventually developed was ‘inoculation’ or ‘variolation’, which involved taking a sample from an infected individual with a lancet, or sharp instrument, and piercing the skin of an uninfected subject. Such treatments aided a subject in developing protective immunity against subsequent infections. The first individual to publish on such treatment in 1796 was Dr. Edward Jenner, who is now credited with discovery of the smallpox vaccine.

Since that time vaccines have developed dramatically, with vaccines being used for many common diseases, including for example, Chickenpox (Varicella), Diphtheria, Flu (Influenza), Hepatitis A and B, Hib, Measles, Mumps, Polio, Pneumococcal, Rotavirus, Rubella, Tetanus and Whooping Cough (Pertussis). In addition to prevention of diseases by infectious agents, vaccines are also being developed for other non-infectious diseases, such as cancer. Particularly in this latter respect, the lines have become blurred with respect to the prevention of a cancer, and the treatment of cancer, wherein a body's immune system can be harnessed to help treat the disease (instead of merely preventing a disease).

The present invention overcomes shortcomings of current commercial vaccines, and further provides additional unexpected benefits.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention provides viral vectors comprising a recombinant virus which expresses an immunomodulatory protein and a target antigen unrelated to said recombinant virus. Advantageously, within certain embodiments the viral vectors can also express natural viral molecules that may function as protective antigens or adjuvants to boost the innate immune system of the host and induce a robust adaptive response against the target antigen. Such recombinant viruses can be utilized to prevent (e.g., as a vaccine) or treat disease due to a pathogenic agent.

Within one aspect of the invention the target antigen is from a pathogenic agent such as a bacterium, parasite (e.g., malaria), or virus. However, pathogenic agents can also include cells such as cancer cells (or antigens on those cells, such as tumor antigens). Within various embodiments of the invention the target antigen may be expressed on the surface of the recombinant viral vector, and/or secreted by the recombinant viral vector.

Within another aspect of the invention, the recombinant viral vector is derived from a virus such as an adenovirus, herpes simplex virus (HSV), influenza virus, rhabdovirus (e.g. vesicular stomatitis virus (VSV)) and pox viruses such as vaccinia virus. Within preferred embodiments of the invention, if the pathogenic agent is a virus, the recombinant viral vector may be derived from a virus different from the pathogenic agent. Within various embodiments of the invention the recombinant virus may be replication competent, replication incompetent, oncolytic and/or non-oncolytic.

Within other aspects of the invention the recombinant viral vector expresses an immunomodulatory protein such as a cytokine, chemokine, costimulatory molecule, and/or an active fragment of any one or more of these.

Within yet other aspects of the invention a vaccine is provided comprising one of the aforementioned recombinant viral vectors, as well as methods for treating and/or preventing diseases caused by a pathogenic agent comprising the step of administering a recombinant viral vector as described herein.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary, to employ concepts of the various patents, applications and publications as identified herein to provide yet further embodiments. Other features, objects and advantages will be apparent from the description, the drawings, and the claims.

### BRIEF DESCRIPTION OF THE DRAWINGS

FIG. 1 is a diagramatic illustration of one embodiment of a recombinant viral vaccine.

FIG. 2 is a representative list of protective antigens.

### DETAILED DESCRIPTION OF THE INVENTION

The present invention may be understood more readily by reference to the following detailed description of preferred embodiments of the invention and the Examples included herein.

The term “virus” refers generally to a class of infectious agents characterized by their small size (historically they were ‘filterable’), and simple organization (generally composed of either DNA or RNA and surrounded by a protein coat or membranous envelope. Representative examples of viruses which are suitable for the construction of the recombinant viral vectors described herein include, without limitation, adenovirus, coxsackievirus, H-1 parvovirus, herpes simplex virus (HSV), influenza virus, measles virus, Myxoma virus, Newcastle disease virus, parvovirus picornavirus, reovirus, rhabdovirus (e.g. vesicular stomatitis virus (VSV)), paramyxovirus such as Newcastle disease virus, picornavirus such as poliovirus or Seneca valley virus, pox viruses such as vaccinia virus (e.g. Copenhagen, Indiana Western Reserve, and Wyeth strains), reovirus, or retrovirus such as murine leukemia virus. Further representative examples are described in: U.S. Pat. Nos. 8,147,822 and 9,045,729 (rhabdovirus/VSV); U.S. Pat. No. 9,272,008 (Measles virus); U.S. Pat. Nos. 7,223,593, 7,537,924, 7,063,835, 7,063,851, 7,118,755, 8,216,564, 8,277,818, and 8,680,068 (herpes virus vectors); and U.S. Pat. No. 8,980,246 (vaccinia virus), all of which are incorporated by reference in their entirety.

The term “immunomodulatory protein” refers to a protein that is capable of altering or modulating the immune system of a subject. Immunomodulatory proteins may be derived from naturally occurring proteins such as cytokines, chemokines, and/or costimulatory molecules (e.g., recombinantly produced from sequences encoding the entire molecule or active fragments thereof).

Representative examples of immunomodulatory proteins include: a) cytokines (or an active fragment thereof) such as IL-1, IL-2, IL-4, IL-5, IL-6, IL-10, IL-12, IL-15, IL-18, GM-CSF, and interferon gamma; b) chemokines (or an active fragment thereof) such as IL-8, SDF-1α, MCP1, MCP2, MCP3 and MCP4 or MCP5, RANTES, MIP-5, MIP-3, eotaxin, MIP-1α, MIP-1β, CMDC, TARC, LARC, or SLC; and/or c) costimulatory molecule (or an active fragment thereof) such as CD80, CD86, ICAM-1, LFA-3, C3d, CD40-L, or Flt3L. Within various embodiments of the invention the immunomodulatory protein can be either secretable or linked to the surface of the recombinant viral vector (e.g., through a viral surface protein).

Within various embodiments the immunomodulatory protein is an immune checkpoint regulator (e.g., an agonist of an immune cell stimulatory receptor such as an agonist of BAFFR, BCMA, CD27, CD28, CD40, CD122, CD137, CD226, CRTAM, GITR, HVEM, ICOS, DR3, LTBR, TACI and/or OX40, or, an antagonist of an inhibitory signal of an immune cell, such as an antagonist of A2AR, BTLA, B7-H3, B7-H4, CTLA4, GAL9, IDO, KIR, LAG3, PD-1, TDO, TIGIT, TIM3 and/or VISTA (see, e.g., “Immune Checkpoint Inhibitors in Cancer” 2019 Elsevier Inc., ISBN-13: 978-0323549486, which is incorporated by reference in its entirety).

The term “target antigen” refers to an antigen from a pathogenic agent which is responsible for a disease state (or initiation of a disease state) in a subject. As noted above, common pathogenic agents include bacterial, viral, or parasitic agents, but can also include disease states in a subject (e.g., cancer). Representative examples of pathogenic agents from which target antigens can be selected include: a) bacteria from genus such as Bacillus, Bartonella, Bordatella, Borrelia, Brucella, Campylobacter, Chlamydia and Chlamydophlia, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Streptococcus, Treponema, Ureaplasma, Vibrio and Yersinia; b) virus from family such as Adenoviridae, Arenaviridae, Bunyaviridae, Calciviridae, Coronaviridae, Filoviridae, Flaviviridae, Hepadnaviridae, Hepeviridae, Herpesviridae, Orthomyxoviridae, Paramyxoviridae, Parvoviridae, Papillomaviridae, Picornoviridae, Polyomaviridae, Poxviridae, Reoviridae, Retroviridae, Rhabdoviridae, and Togaviridae; and c) parasites, including for example protozoans such as amoeba, Giardia lamblia, Leshmania spp., Plasmodium spp., Toxoplasma gondii, Trichomonas vaginalis, and Trypanosoma spp.

“Tumor antigen” or “tumor antigens” as utilized herein refers to antigens that presented by MHC class I or class II molecules on the surface of tumor cells. Antigens which are found only on tumor cells are referred to as “Tumor Specific Antigens” or “TSAs”, while antigens that are presented by both tumor cells and normal cells are referred to as “Tumor Associated Antigens” or “TAAs”. Representative examples of tumor antigens include, but are not limited to AIM-2, AIM-3, ART1, ART4, BAGE, β1,6-N, β-catenin, B-cyclin, BM11, BRAF, BRAP, C13orf24, C6orf153, C9orf112, CA-125, CABYR, CASP-8, cathepsin B, Cav-1, CD74, CDK-1, CEAmidkin, COX-2, CRISP3, CSAG2, CTAG2, CYNL2, DHFR, E-cadherin, EGFRvIII, EphA2/Eck, ESO-1, EZH2, Fra-1/Fosl 1, FTHL17, GAGE1, Ganglioside/GD2, GLEA2, Glil, GnT-V, GOLGA, gp75, gplOO, HER-2, HSPH1, IL13Ralpha, IL13Ralpha2, ING4, Ki67, KIAA0376, Ku70/80, LDHC, LICAM, Livin, MAGE-A1, MAGE-2, MAGE-A3, MAGE-B6, MAPPK1, MART-1, MICA, MRP-3, MUC-1, MUM-1, Nestin, NKTR, NLRP4, NSEP1, NY-ES-01, OLIG2, p53, PAP, PBK, PRAME, PROX1, PSA, PSCA, PSMA, ras, RBPSUH, RTN4, SART1, SART2, SART3, SOX10, SOX11, SOX2, SPANXA1, SSX2, SSX4, SSX5, Survivin, TNKS2, TPR, TRP-1, TRP-2, TSGA10, TSSK6, TULP2, Tyrosinase, U2AF1L, UPAR, WT-1, XAGE2, and ZNF165.

Within certain embodiments of the invention CEACAM6, CEACAM5, NY-ESO-1, and EpCAM are utilized as surface markers for tumor targeting. Briefly, CEACAM6 and CEACAM5 (carcinoembryonic antigen-related cell adhesion molecule) are cell surface glycoproteins which function as intercellular adhesion molecules. EpCAM (epithelial cell adhesion molecule) is a transmembrane glycoprotein which mediates homotypic cell-cell adhesion. EpCAM is highly expressed in most epithelial-derived neoplasms and has been used as a diagnostic and prognostic marker for a variety of carcinomas. EpCAM plays a role in carcinogenesis by promoting cell proliferation and metastasis and by transcriptionally upregulating oncogenes c-myc and cyclin A/E. NY-ESO-1 (New York esophageal squamous cell carcinoma 1) is well known cancer testis antigen with re-expression in numerous cancer types.

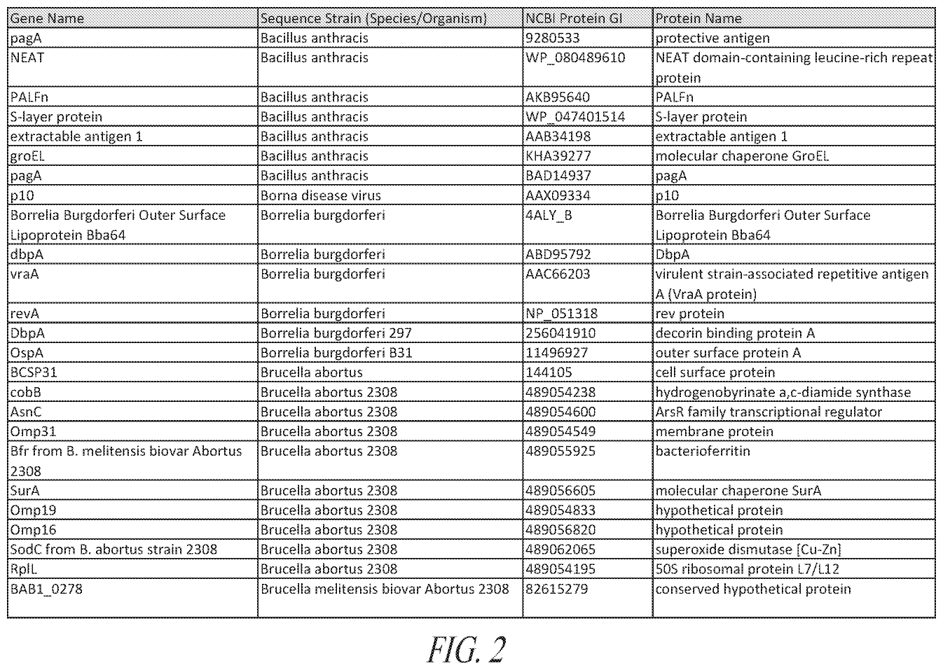

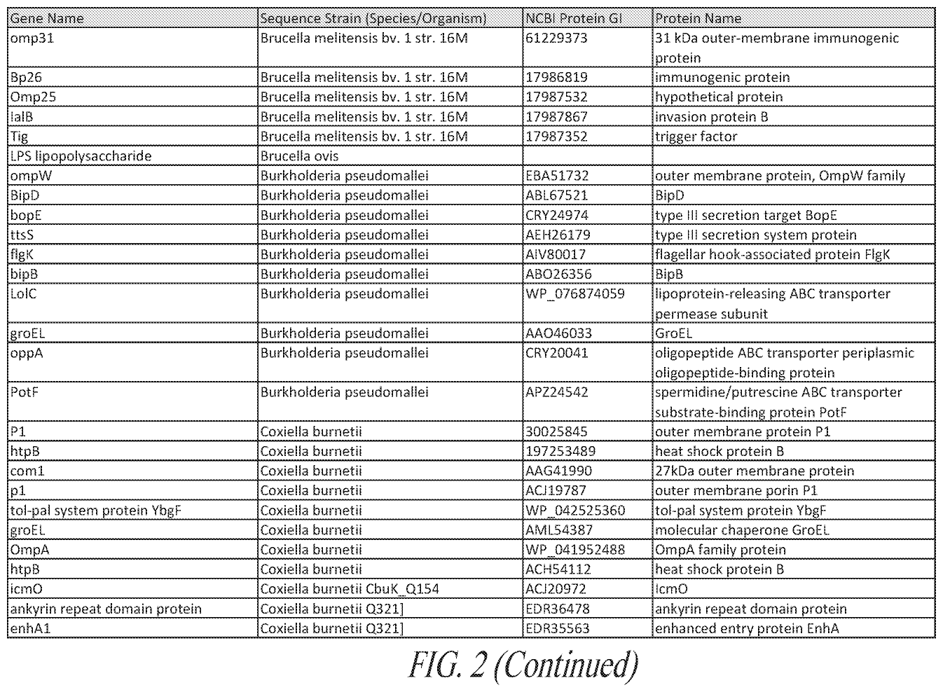

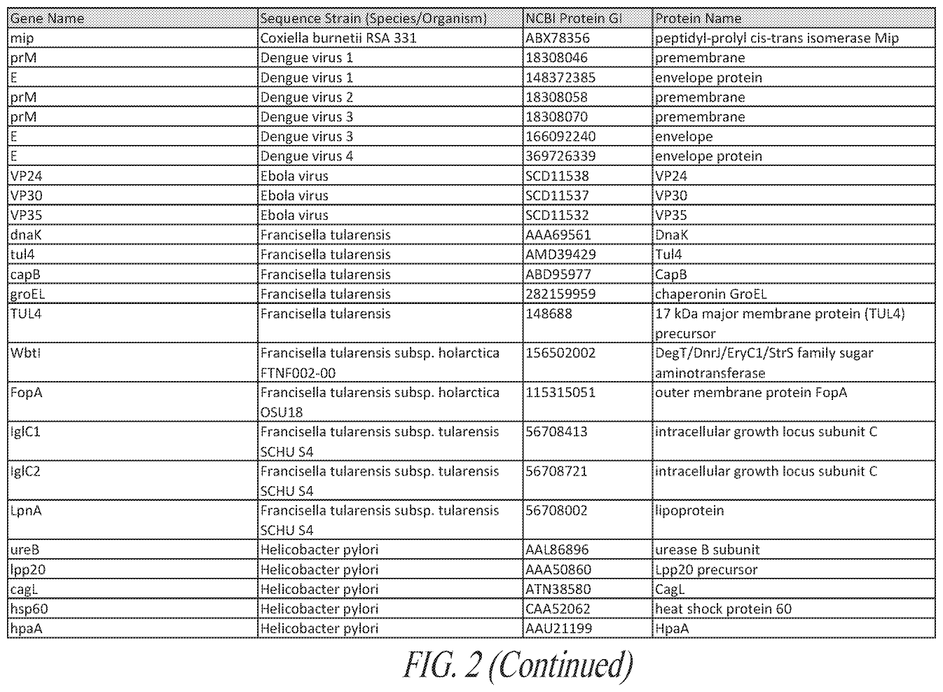

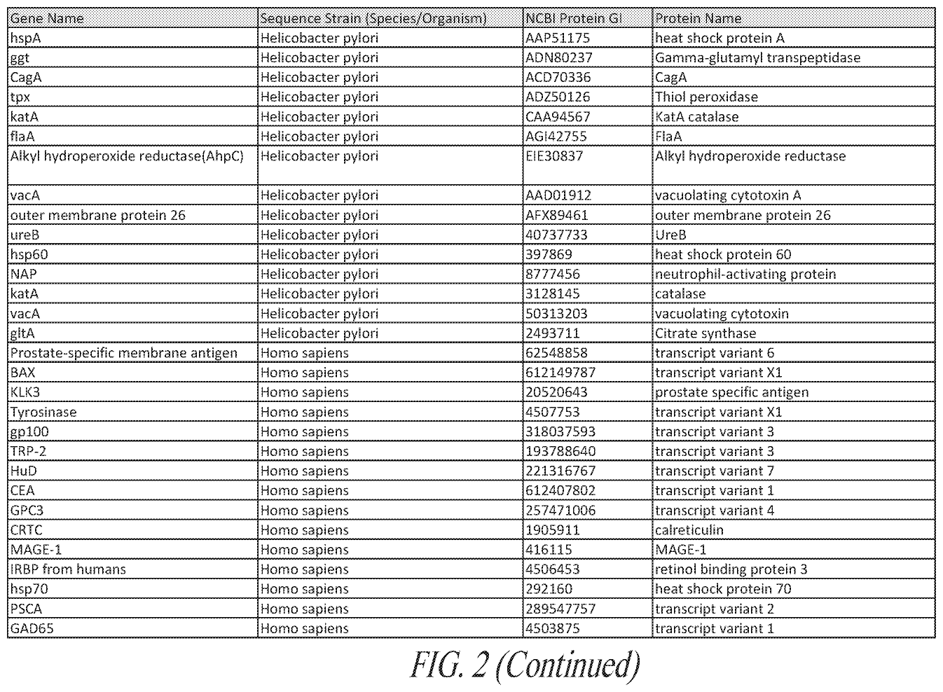

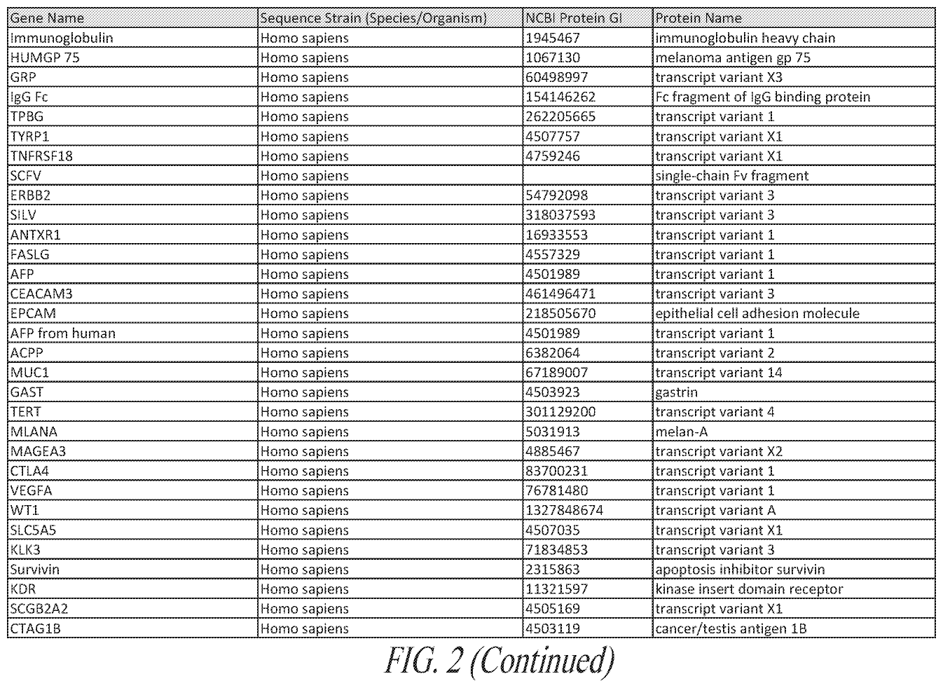

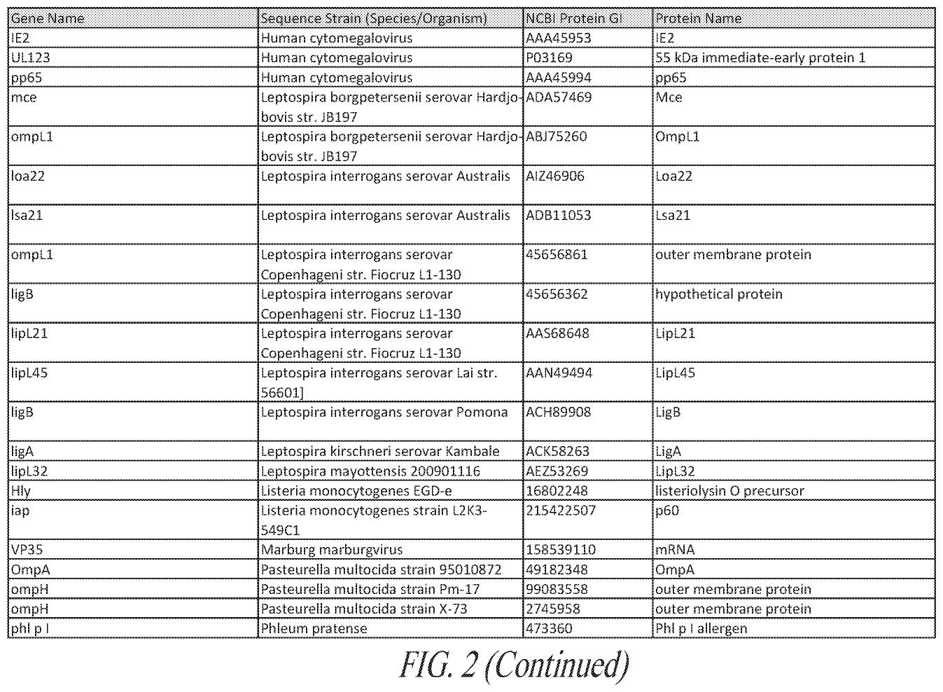

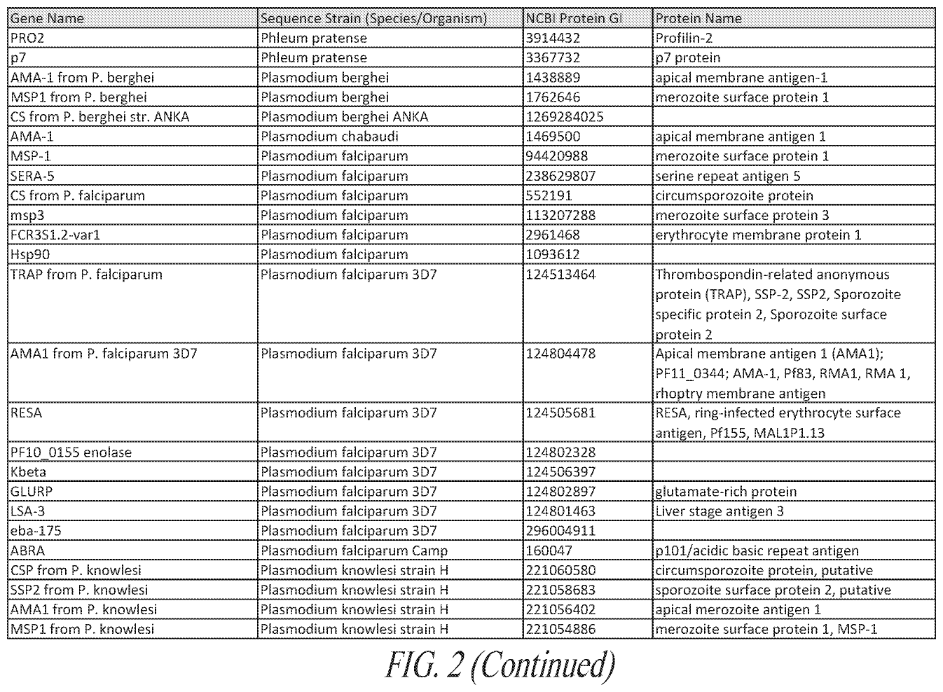

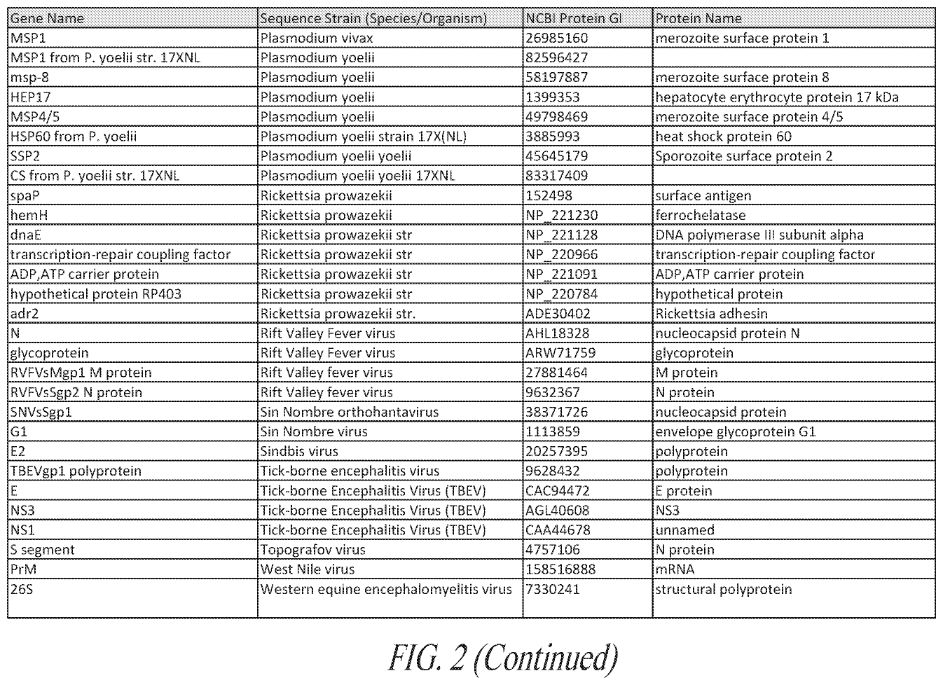

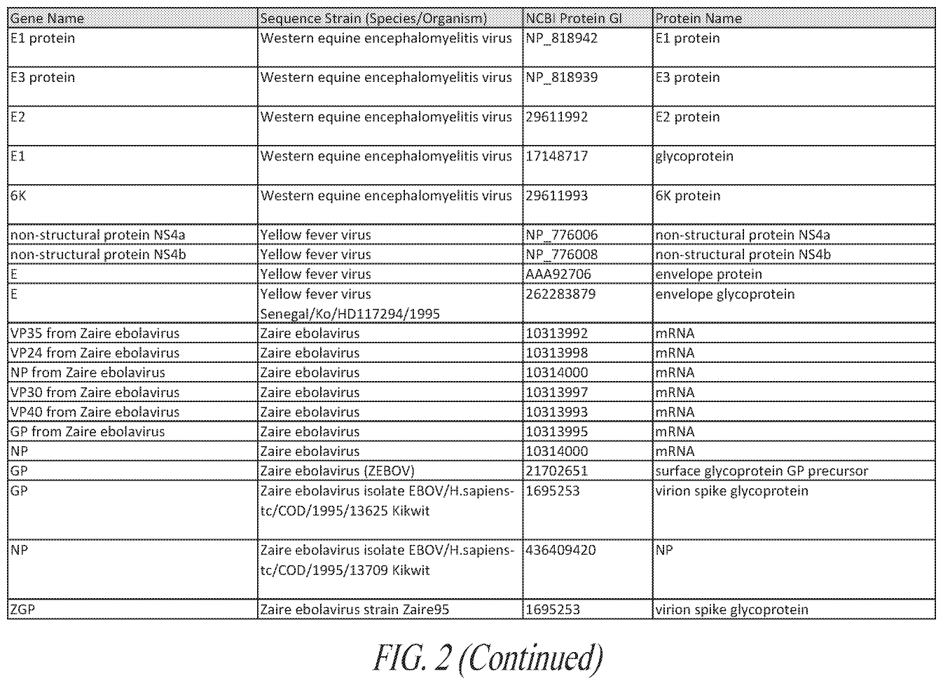

The term “protective antigen” refers to viral antigens that are specifically targeted by the acquired immune system of the host, and when introduced into the host body, are able to stimulate the production of antibodies and/or cell-mediated immunity against certain pathogens or the causes of other diseases. Representative examples of protective antigens include, but are not limited to, those disclosed in Yang et al. Nucleic Acids Research, 2011; 39(suppl_1):D1073-D1078, which is herein incorporated by reference in its entirety. Within one embodiment a representative list of protective antigens is set forth in FIG. 2.

“Treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

The term “cancer” refers to a disease state caused by uncontrolled or abnormal growth of cells in a subject. Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

In order to further understand the various aspects of the invention provided herein, the following sections are provided below: A. Recombinant Viral Vectors; B. Target Antigens and Immunomodulatory Proteins; C. Therapeutic Compositions/Vaccines; and D. Administration.

### A. Recombinant Viral Vectors

As noted above, the present invention provides a viral vector comprising a recombinant virus which expresses an immunomodulatory protein and a target antigen unrelated to the recombinant virus. Representative examples of viruses which are suitable for the construction of the recombinant viral vectors described herein include, without limitation, adenovirus, coxsackievirus, H-1 parvovirus, herpes simplex virus (HSV), influenza virus, measles virus, Myxoma virus, Newcastle disease virus, parvovirus picornavirus, reovirus, rhabdovirus (e.g. vesicular stomatitis virus (VSV)), paramyxovirus such as Newcastle disease virus, picornavirus such as poliovirus or Seneca valley virus, pox viruses such as vaccinia virus (e.g. Copenhagen, Indiana Western Reserve, and Wyeth strains), reovirus, or retrovirus such as murine leukemia virus.

Within a preferred embodiment of the invention, the recombinant viral vector is derived from a Herpes Simplex Virus. Briefly, Herpes Simplex Virus (HSV) 1 and 2 are members of the Herpesviridae family, which infects humans. The HSV genome contains two unique regions, which are designated unique long (U_{L}) and unique short (U_{S}) region. Each of these regions is flanked by a pair of inverted terminal repeat sequences. There are about 75 known open reading frames. The viral genome has been engineered to develop viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic.

Suitable HSV may be derived from either HSV-1 or HSV-2, including any laboratory strain or clinical isolate. In some embodiments, the HSV may be or may be derived from one of laboratory strains HSV-1 strain 17, HSV-1 strain F, HSV-1 strain KOS, HSV-1 strain McKrae, or. HSV-2 strain HG52. In other embodiments, it may be of or derived from non-laboratory strain JS-1. Other suitable HSV-1 viruses include HrrR3 (Goldstein and Weller, J. Virol. 62, 196-205, 1988), G207 (Mineta et al. Nature Medicine. 1(9):938-943, 1995; Kooby et al. The FASEB Journal, 13(11):1325-1334, 1999); G47Delta (Todo et al. Proceedings of the National Academy of Sciences. 2001; 98(11):6396-6401); HSV 1716 (Mace et al. Head& Neck, 2008; 30(8):1045-1051; Harrow et al. Gene Therapy. 2004; 11(22):1648-1658); HF10 (Nakao et al. Cancer Gene Therapy. 2011; 18(3):167-175); NV1020 (Fong et al. Molecular Therapy, 2009; 17(2):389-394); T-VEC (Andtbacka et al. Journal of Clinical Oncology, 2015: 33(25):2780-8); J100 (Gaston et al. PloS one, 2013; 8(11):e81768); M002 (Parker et al. Proceedings of the National Academy of Sciences, 2000; 97(5):2208-2213); NV1042(Passer et al. Cancer Gene Therapy. 2013; 20(1):17-24); G207-IL2 (Carew et al. Molecular Therapy, 2001; 4(3):250-256); rQNestin34.5 (Kambara et al. Cancer Research, 2005; 65(7):2832-2839); G47A-mIL-18 (Fukuhara et al. Cancer Research, 2005; 65(23):10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat1/3 Inhibitors with Oncolytic Herpes Virus”, all of the above of which are incorporated by reference in their entirety.

The HSV vector may have modifications, mutations, or deletion of at least one γ34.5 gene. In some embodiments, both genes are deleted, mutated or modified. In other embodiments, one is deleted, and the other is mutated or modified. Either native γ34.5 gene can be deleted. In one embodiment, the terminal repeat, which comprises γ34.5 gene and ICP4 gene, is deleted. Mutations, such as nucleotide alterations, insertions and deletions may be used to render the gene inexpressible or the product inactive. The γ34.5 gene may be modified with miRNA target sequences in its 3′ UTR. The target sequences bind miRNAs that are expressed at lower levels in tumor cells than in their normal counterparts. In some embodiments, the modified or mutated γ34.5 gene(s) are constructed in vitro and inserted into the HSV vector as replacements for the viral gene(s). When the modified or mutated γ34.5 gene is a replacement of only one γ34.5 gene, the other γ34.5 is deleted. The γ34.5 gene may comprise additional changes, such as having an exogenous promoter. Within further embodiments, the γ34.5 gene can be translationally regulated, e.g., via the addition of an exogenous 5′ UTR such as the rat FGF-2 5′ UTR. This 5′ UTR forms secondary hairpin structures that can be unwound in the presence of sufficient eukaryotic initiation factor (eIF)4E/eIF4F complexes, leading to translation initiation of the mRNA. The eIF4E protein, part of the eIF4F complex, is known to be overexpressed in a variety of cancer types. Within yet other embodiments of the invention, neurovirulence may be prevented without modification of γ34.5 gene by employing mutations which prevent the virus from entering neurons in the first place, for example, by deleting amino acids 31-68 of glycoprotein K.

The HSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP 24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible upon delivery of an inducer or inducible upon a cellular event or particular environment. In particular embodiments, a tumor-specific promoter drives expression of viral genes essential for replication of HSV. In certain embodiments the expression of ICP4 or ICP27 or both is controlled by an exogenous promoter, e.g., a tumor-specific promoter. Exemplary tumor-specific promoters include CEA, CXCR4, TERT, survivin or telomerase; other suitable tumor-specific promoters may be specific to a single tumor type and are known in the art. Other elements may be present. In some cases, an enhancer such as NF-kB/OCT4/SOX2 enhancer is present, for example in the regulatory regions of ICP4 or ICP27 or both. As well, the 5′UTR may be exogenous, such as a 5′UTR from growth factor genes such as FGF.

The HSV may also have genes and nucleotide sequences that are non-HSV in origin. For example, a sequence that encodes one of the aforementioned target antigens, an immunomodulatory protein, a prodrug, a sequence that encodes a cytokine or other immune stimulating factor, a tumor-specific promoter, an inducible promoter, an enhancer, a sequence homologous to a host cell, among others may be in the HSV genome. Exemplary sequences encode IL12, IL15, OX40L, PD-L1 blocker or a PD-1 blocker. For sequences that encode a product, they are operatively linked to a promoter sequence and other regulatory sequences (e.g., enhancer, polyadenylation signal sequence) necessary or desirable for expression.

The regulatory region of viral genes may be modified to comprise response elements that affect expression. Exemplary response elements include response elements for NF-κB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

Representative examples of HSV vectors are described in PCT/2017/018539, PCT/US2017/030308, PCT/US2017/044993, PCT/US2018/061687, U.S. Ser. No. 15/374,893, and U.S. Ser. No. 15/588,616, all of which are incorporated by reference in their entirety.

### B. Target Antigens and Immunomodulatory Proteins

As noted above, the present invention provides recombinant viral vectors which express a desired target antigen and an immunomodulatory protein (both of which are discussed in more detail above). Within various embodiments of the invention, the target antigen and/or immunomodulatory protein may be secreted from the recombinant viral vector, and/or expressed on the viral surface (e.g., through fusion with a viral surface protein).

For example, within one embodiment of the invention, HSV recombinant viral vectors are generated with a deletion in the ectodomains of an envelope protein (e.g., gC, gD or gG are readily generated by homologous recombination technology. Specifically, viral mutagenesis is performed using a lambda Red-mediated recombineering system implemented on the HSV-1 genome cloned into a bacterial artificial chromosome (BAC). Utilizing such methods, a desired target antigen or immunomodulatory protein can be linked to truncated gC, gD or gG for expression on the surface of the viral vector.

Within yet other embodiments of the invention, HSV recombinant viral vectors can also be generated by inserting the target antigen and/or immunomodulatory protein into the ectodomain of a viral envelope protein without any truncation of the viral envelope protein.

Representative viral vectors and sites for insertion of target antigens and/or immunomodulatory proteins are also described in PCT Application No. PCT/US2017/030308, filed Apr. 29, 2017, which is hereby incorporated by reference in its entirety.

### C. Therapeutic Compositions/Vaccines

As noted above, the present invention provides for vaccines comprising one of the recombinant viral vectors described herein, along with a pharmaceutically acceptable excipient. The phrase “pharmaceutically acceptable carrier” is meant to encompass any carrier, diluent or excipient that does not interfere with the effectiveness of the biological activity of the virus and that is not toxic to the subject to whom it is administered (see generally Remington: The Science and Practice of Pharmacy, Lippincott Williams & Wilkins; 21st ed. (May 1, 2005 and in The United States PharmacopE1A: The National Formulary (USP 40-NF 35 and Supplements).

In the case of the vaccines described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioadsorbable matrix materials, implantation elements containing the virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethyleneglycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the HSV to a target cancer cell will preferably not induce an immune response in the individual (subject) receiving the composition (and will preferably be administered without undue toxicity).

The compositions provided herein can be provided at a variety of concentrations. For example, dosages of recombinant virus can be provided which ranges from about 10⁶ to about 10⁹ pfu/ml. Within further embodiments, the dosage can range from about 10⁶ to about 10⁸ pfu/ml, with up to 4 ms being injected into a patient with large lesions (e.g., >5 cm) and smaller amounts (e.g., up to 0.1 mls) in patients with small lesions (e.g., <0.5 cm) every 2-3 weeks, of treatment.

Within certain embodiments of the invention, lower dosages than standard may be utilized. Hence, within certain embodiments less than about 10⁶ pfu/ml (with up to 4 mls being injected into a patient every 2-3 weeks) can be administered to a patient.

The compositions may be stored at a temperature conducive to stable shelf-life and includes room temperature (about 20° C.), 4° C., −20° C., −80° C., and in liquid N2. Because compositions intended for use in vivo generally don't have preservatives, storage will generally be at colder temperatures. Compositions may be stored dry (e.g., lyophilized) or in liquid form.

### D. Administration

In addition to the compositions described herein, as noted above the present invention provides methods for vaccinating a subject against a pathogenic agent, comprising the step of administering to a subject an effective amount of one of the recombinant viral vectors described herein.

The terms “effective dose” and “effective amount” refers to amounts of the recombinant viral vector that are sufficient to prevent a subject from infection from a virulent pathogenic agent (e.g., infection by a bacteria, virus or parasite as described herein). Within other embodiments, the term “effective dose” and “effective amount” refers to amounts of the recombinant viral vector that are sufficient to effect treatment of a targeted cancer, e.g., amounts that are effective to reduce a targeted tumor size or load, or otherwise hinder the growth rate of targeted tumor cells.

More particularly, such terms refer to amounts of virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer.

Effective amounts may vary according to factors such as the subject's disease state, age, gender, and weight, as well as the pharmaceutical formulation, the route of administration, and the like, but can nevertheless be routinely determined by one skilled in the art.

The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Within preferred embodiments of the invention, the vaccine is administered by a variety of routes depending on the type of vaccine (e.g. intramuscularly, subcutaneous, or transdermal).

The optimal or appropriate dosage regimen of the virus is readily determinable within the skill of the art, by the attending physician based on patient data, patient observations, and various clinical factors, including for example a subject's size, body surface area, age, gender, and the particular virus being administered, the time and route of administration, the type of cancer being treated, the general health of the patient, and other drug therapies to which the patient is being subjected.

The following are additional exemplary embodiments of the present disclosure:

1) A recombinant viral vector, comprising a recombinant virus which expresses an immunomodulatory protein and a target antigen unrelated to said recombinant virus. As used herein it should be understood that a target antigen is “unrelated” to a recombinant virus if the target antigen is derived from a different species than the recombinant virus. Within certain embodiments the target antigen is from a bacterium. Within related embodiments, the target antigen is a protective antigen, representative examples of which are set forth in FIG. 2. Within other embodiments, the protective antigen is derived from one of the organisms set forth in FIG. 2. Target antigens as described herein may include the entire protein sequence, or, fragments thereof (e.g., immunologically active fragments of the antigens set forth in FIG. 2).

2) The viral vector according to embodiment 1 wherein said virus is selected from the group consisting of an adenovirus, a vaccinia virus, and a herpes virus.

3) The viral vector according to embodiments 1 or 2 wherein said virus is a replication competent virus. Within related embodiments the virus may be attenuated (e.g., through UV), or, is conditionally regulated (e.g., it replicates principally in tumor tissue but not in normal tissue).

4) The viral vector according to embodiments 1 or 2 wherein said virus is a replication incompetent virus.

5) The viral vector according to any one of embodiments 1 to 4 wherein said target antigen is expressed on the surface of the virus. Within other embodiments of the invention, the target antigen may be secreted from the viral vector.

6) The viral vector according to embodiment 5 wherein said target antigen is fused to a viral glycoprotein. Within yet other embodiments the immunomodulatory protein is fused to a viral glycoprotein. Within yet other embodiments of the invention a target antigen (e.g., an antigen as set forth in FIG. 2 or from an organism as set forth in FIG. 2) may be fused or otherwise combined with an envelope glycoprotein (see, e.g., PCT/US2018/061687 which is incorporated by reference in its entirety).

7) The viral vector according to embodiment 6 wherein said recombinant virus is a herpes virus and said viral glycoprotein is an envelope protein is selected from the group consisting of gB, gC, gD, gE, gG, gI, gJ, gK, gM, gN, UL20, UL24, UL43, UL45, UL56, and US9.

8) The viral vector according to any one of embodiments 1 to 7 wherein said target antigen is an antigen from a virus unrelated to the parent virus of the recombinant viral vector. As utilized herein an ‘unrelated’ virus is a virus of a different species from the recombinant viral vector. Within other embodiments the ‘unrelated’ virus may be from a different Kingdom, Subkingdom, Phylum, Subphylum, Class, Subclass, Order, Suborder, Family, Subfamily, Genus, or, Subgenus of the recombinant viral vector.

9) The viral vector according to any one of embodiments 1 to 7 wherein said target antigen is from a bacterium or a parasite.

10) The viral vector according to any one of embodiments 1 to 7 wherein said target antigen is a tumor antigen.

11) The viral vector according to any one of embodiments 1 to 7 wherein said recombinant viral vector expresses multiple target antigens. Within certain embodiments of the invention, the target antigens can be derived from different variants or strains of an organism (e.g., from different strains of influenza)

12) The viral vector according to any one of embodiments 1 to 11, wherein said immunomodulatory protein is a cytokine, a chemokine, a costimulatory molecule, or an active fragment of any of these. Representative examples of immunomodulatory proteins include IL-12, IL-15, IL-15Ralpha. Other representative examples include immune checkpoint regulators, illustrative examples of which include checkpoint regulators (e.g., peptides or antibodies against PD-1, PD-1, VISTA, TIM3, TIGIT), TNF-alpha, TLR agonists, TGF-b antagonists, and the OX40 ligand.

13) The viral vector according to embodiment 11 wherein said immunomodulatory protein is secreted from said viral vector.

14) A vaccine, comprising the viral vector according to any one of embodiments 1 to 13, along with a pharmaceutically acceptable excipient.

15) A method for vaccinating a subject against a pathogenic agent, comprising the step of administering an effective amount of vaccine according to embodiment 14 which expresses a target antigen from said pathogenic agent.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in the written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

### EXAMPLES

All constructs are generated using standard molecular cloning and recombineering techniques, familiar to those skilled in the art.

### Example 1

### Design of a Recombinant Viral Vaccine with Surface-Bound Tumor Antigen

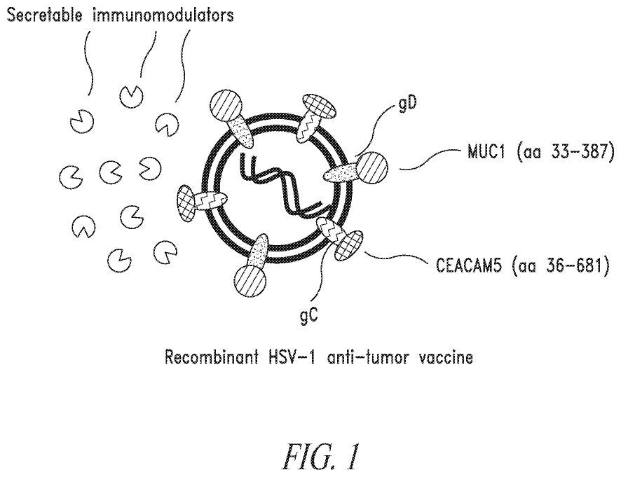

Recombinant viral vaccines may be engineered in which CEACAM5 and/or MUC1 proteins, or fragments thereof, are fused to the surface of a HSV-1 virus particle, as depicted in simplified form in FIG. 1. In one embodiment, CEACAM5 and/or MUC1 fragments lacking the signal peptide and the transmembrane and cytoplasmic domains are fused to an HSV-1 surface glycoprotein. For example, amino acids 36-681 of the CEACAM5 protein (SEQ ID NO:1), comprising the extracellular domain, is fused in-frame to the extracellular domain of glycoprotein C (gC) or glycoprotein D (gD) at a location downstream of the signal peptide. In another embodiment, amino acids 33-387 of the MUC1 protein (SEQ ID NO:2), comprising the extracellular domain, is fused in-frame to the extracellular domain of gC or gD at a location downstream of the signal peptide.

### Example 2

### Design of a Recombinant Viral Vaccine that Induces Secretion of Tumor Antigen

In another embodiment of a recombinant viral vaccine, the transgenes encoding CEACAM5 and/or MUC1 are cloned into the HSV-1 genome such that the recombinant protein products are expressed and secreted by the infected cells. The expression cassette(s) encoding the extracellular domain of CEACAM5 and/or MUC1 (including the signal peptide) are inserted into the HSV-1 genome in one of the following locations: between UL3 and UL4, between UL50 and UL51, between US1 and US2, between UL7 and UL8, between UL10 and UL11, between UL15 and UL18, between UL21 and UL22, between UL26 and UL27, between UL35 and UL36, between UL40 and UL41, between UL45 and UL46, between UL55 and UL56, or between Us9 and Us10.

For the CEACAM5 antigen, a nucleic acid encoding amino acids 1-681 (SEQ ID NO:3), comprising the extracellular domain of CEACAM5 (including the signal peptide) is used, while for the MUC1 antigen, a nucleic acid encoding amino acids 1-287 (SEQ ID NO:4), comprising the extracellular domain of MUC1 (including the signal peptide) is used. An additional recombinant virus is constructed that does not express any transgenes in order to be used as a negative control.

### Example 3

### Design of a Recombinant Viral Vaccine with Surface-Bound Bacterial Antigen

In one embodiment of a recombinant viral vaccine against an infectious disease, the OspA lipoprotein from Borrelia burgdorferi (the causative agent of Lyme disease) is expressed on the surface of the HSV-1 virus particle. In this embodiment, the full-length OspA protein lacking the signal peptide (SEQ ID NO: 5) is fused in-frame to an HSV-1 surface glycoprotein, such as glycoprotein C (gC) or glycoprotein D (gD) at a location downstream of the signal peptide.

### Example 4

### Design of a Recombinant Viral Vaccine that Induces Secretion of Bacterial Antigen

In another embodiment of a recombinant viral vaccine against an infectious disease, a nucleic acid encoding the OspA lipoprotein from Borrelia burgdorferi (the causative agent of Lyme disease) is cloned into the HSV-1 genome such that the protein product is expressed and secreted by the infected cells. An expression cassette encoding the entire OspA protein comprising amino acids 1-273 (SEQ ID NO:6) is inserted into the HSV-1 genome in one of the following locations: between UL3 and UL4, between UL50 and UL51, between US1 and US2, between UL7 and UL8, between UL10 and UL11, between UL15 and UL18, between UL21 and UL22, between UL26 and UL27, between UL35 and UL36, between UL40 and UL41, between UL45 and UL46, between UL55 and UL56, or between Us9 and Us10.

### Example 5

### Analysis of Recombinant Viral Vaccines

All recombinant viruses are purified using a combination of gel filtration, centrifugation, tangential flow filtration or other methods. An animal model is utilized for testing each vaccine candidate. Virus doses ranging from 10⁷-10⁹ pfu/mouse are used to immunize BALB/c and C57 B/6 mice via subcutaneous, intramuscular, intraperitoneal and/or intradermal injections. A range of 1-3 doses is tested, with 1-week intervals between doses. Serum is collected from immunized mice at different timepoints (pre-immunization, 5 days, 7 days, 14 days, 21 days and 28 days post-immunization). ELISA is used to measure the humoral immune response to the immunizing antigen(s). Based on ELISA results and the serum antibody titer detected, spleen cells are collected for testing the cellular immune response using IFN-gamma and IL-2 ELISPOT assays. Immunized mice are challenged with the infectious agent (in the case of antigens derived from pathogens) or with a tumor cell line expressing a tumor associated antigen (in case of TAA-based vaccines).

What is claimed is:

1. A recombinant viral vector, comprising a recombinant virus which expresses an immunomodulatory protein and a target antigen unrelated to said recombinant virus.

2. The viral vector according to claim 1 wherein said virus is selected from the group consisting of an adenovirus, a vaccinia virus, and a herpes virus.

3. The viral vector according to claims 1 or 2 wherein said virus is a replication competent virus.

4. The viral vector according to claims 1 or 2 wherein said virus is a replication incompetent virus.

5. The viral vector according to any one of claims 1 to 4 wherein said target antigen is expressed on the surface of the virus.

6. The viral vector according to claim 5 wherein said target antigen is fused to a viral glycoprotein.

7. The viral vector according to claim 6 wherein said recombinant virus is a herpes virus and said viral glycoprotein is an envelope protein selected from the group consisting of gB, gC, gD, gE, gG, gI, gJ, gK, gM, gN, UL20, UL24, UL43, UL45, UL56, and US9.

8. The viral vector according to any one of claims 1 to 7 wherein said target antigen is an antigen from a virus unrelated to the parent virus of the recombinant viral vector.

9. The viral vector according to any one of claims 1 to 7 wherein said target antigen is from a bacterium or a parasite.

10. The viral vector according to any one of claims 1 to 7 wherein said target antigen is a tumor antigen.

11. The viral vector according to any one of claims 1 to 7 wherein said recombinant viral vector expresses multiple target antigens.

12. The viral vector according to any one of claims 1 to 11, wherein said immunomodulatory protein is a cytokine, a chemokine, a costimulatory molecule, or an active fragment of any of these.

13. The viral vector according to claim 11 wherein said immunomodulatory protein is secreted from said viral vector.

14. A vaccine, comprising the viral vector according to any one of claims 1 to 13, along with a pharmaceutically acceptable excipient.

15. A method for vaccinating a subject against a pathogenic agent, comprising the step of administering an effective amount of vaccine according to claim 15 which expresses a target antigen from said pathogenic agent.
